(19) United States
(12) Patent Application Publication
(10) Pub. No.: US 2008/0262585 A1
(43) Pub. Date: Oct. 23, 2008

(54) IMPLANTABLE MEDICAL ELECTRICAL LEAD AND CONNECTOR ASSEMBLY

(75) Inventors: James A. Alexander, Shorewood, MN (US); James M. Olsen, Plymouth, MN (US); Chad Q. Cai, Woodbury, MN (US); Richard T. Stone, Minneapolis, MN (US); James G. Skakoon, St. Paul, MN (US); Kristin J. Schneider, St. Michael, MN (US)

Correspondence Address: CAMPBELL NELSON WHIPPS, LLC, 408 ST. PETER STREET, SUITE 240, ST. PAUL, MN, 55102, US

(73) Assignee: MEDTRONIC, INC., Minneapolis, MN (US)

(21) Appl. No.: 11/737,915

(22) Filed: Apr. 20, 2007

(51) Int. Cl. A61N 1/362

(52) U.S. Cl. 607/122

(57) ABSTRACT

An implantable medical system that includes an implantable electrical lead having a lead body having a connector portion wherein at least the connector portion of the lead body has an axial cross section that is substantially non-circular; and a connector block that is configured to be operably coupled to the implantable electrical lead, wherein the connector block includes a lumen having an inner surface that is configured to be complementary to the outer surface of at least the connector portion of the lead body. Leads and connector blocks are also discussed.

### FIELD

Implantable medical electrical lead and connector assembly, more specifically, an implantable medical electrical lead and connector assembly having non-circular axial cross sections.

### BACKGROUND

The medical device industry produces a wide variety of electronic and mechanical devices for treating medical conditions. Commonly used neuromodulators include an implantable signal generator and at least one lead. Such devices are commonly utilized to treat numerous conditions in various portions of the body.

Magnetic resonance imaging (MRI) is commonly used to diagnose many disorders and conditions in many parts of the body. MRI scans utilize strong magnetic fields to produce diagnostic images. Concerns have arisen regarding possible undesirable interactions between the environment created during an MRI scan and implantable medical devices. Implantable medical devices and components thereof are being fabricated to alleviate any possible issues in an MRI environment. However, without a lockout non-MRI safe components can be compatible with MRI safe components.

Therefore, there remains a need for MRI safe implantable medical devices and components thereof that can only be used with other MRI safe components.

### BRIEF SUMMARY

An implantable electrical lead comprising: a lead body having a proximal portion, a distal portion, and a connector portion wherein at least the connector portion of the lead body has an outer surface and an axial cross section that is substantially non-circular; at least one electrode located at the distal portion of the lead body; at least one electrical contact located at the proximal portion of the lead body; and at least one conductive element that electrically couples the at least one electrode to the at least one electrical contact.

An implantable medical device comprising: a connector block that is configured to be operably coupled to an implantable electrical lead, wherein the connector block comprises: a lumen comprising an inner surface and having an axial cross section that is substantially non-circular, and being configured to allow an implantable electrical lead to be inserted; and at least one electrode connector positioned adjacent the inner surface of the lumen, and configured to electrically couple a lead that is inserted into the lumen

An implantable medical system comprising: an implantable electrical lead comprising: a lead body having a proximal portion, a distal portion, and a connector portion wherein at least the connector portion of the lead body has an axial cross section that is substantially non-circular; at least one electrode located at the distal portion of the lead body; at least one electrical contact located within the connector portion of the lead body; and at least one conductive element that electrically couples the at least one electrode to the at least one electrical contact; and a connector block that is configured to be operably coupled to the implantable electrical lead, wherein the connector block comprises: a lumen comprising an inner surface; and at least one electrode connector positioned adjacent the inner surface of the lumen, wherein the at least one electrode connector is configured to be electrically coupled to the at least one electrical contact of the lead, and wherein the inner surface of the lumen has a configuration that is complementary to the outer surface of at least the connector portion of the lead body

### BRIEF DESCRIPTION OF THE DRAWINGS

This disclosure may be more completely understood in consideration of the following detailed description of various embodiments of the disclosure in connection with the accompanying drawings, in which:

FIG. 1 is a diagrammatic representation of a side view of an implantable electrical signal therapy system.

FIG. 2 is a diagrammatic representation of a side view of an implantable electrical signal therapy system.

FIG. 3 is a diagrammatic representation of a portion of a connector block of a representative implantable electrical signal therapy system.

FIG. 4 is a schematic diagram of an active medical device implanted within a human body.

FIG. 5 is a diagrammatic representation of a lead inserted into an implantable signal generator (ISG).

FIG. 6 is a diagrammatic representation of an axial cross section of a portion of a lead.

FIG. 7 is a diagrammatic representation of a portion of a longitudinal cross section of a lead.

FIG. 8 is a diagrammatic representation of an axial cross section of a portion of a lead.

FIG. 9 is a diagrammatic representation of an axial cross section of a portion of a lead.

FIG. 10 is a diagrammatic representation of a portion of a longitudinal cross section of a lead.

FIG. 11 is a diagrammatic representation of an axial cross section of a portion of a lead.

FIG. 12 is a diagrammatic representation of an axial cross section of a portion of a lead.

FIG. 13 is a perspective view of an exemplary lead having a non-circular cross section.

FIG. 14 is a perspective view of an exemplary lead having a non-circular cross section.

FIG. 15 is a perspective view of an exemplary lead having a non-circular cross section.

FIG. 16 is a diagrammatic representation of a lead inserted into an ISG.

FIG. 17 is a representation of an exemplary inner surface of a lumen and outer surface of a lead that are complementary.

FIG. 18 is a representation of an exemplary inner surface of a lumen and outer surface of a lead that are complementary.

The figures provided herein are not necessarily to scale. Like numbers used in the figures refer to like components. However, it will be understood that the use of a number to refer to a component in a given figure is not intended to limit the component in another figure labeled with the same number.

### DETAILED DESCRIPTION

In the following description, reference is made to the accompanying drawings that form a part hereof, and in which are shown by way of illustration several specific embodiments. It is to be understood that other embodiments are contemplated and are part of this disclosure. The following detailed description, therefore, is not to be taken in a limiting sense.

All scientific and technical terms used herein have meanings commonly used in the art unless otherwise specified. The definitions provided herein are to facilitate understanding of certain terms used herein and are not meant to limit the scope of the disclosure.

Unless otherwise indicated, all numbers expressing feature sizes, amounts, and physical properties used in the specification and claims are to be understood as being modified in all instances by the term “about.” Accordingly, unless indicated to the contrary, the numerical parameters set forth in the foregoing specification and attached claims are approximations that can vary depending upon the desired properties sought to be obtained by those skilled in the art utilizing the teachings disclosed herein.

The recitation of numerical ranges by endpoints includes all numbers subsumed within that range (e.g. 1 to 5 includes 1, 1.5, 2, 2.75, 3, 3.80, 4, and 5) and any range within that range.

As used in this specification and the appended claims, the singular forms “a”, “an”, and “the” encompass embodiments having plural referents, unless the content clearly dictates otherwise. As used in this specification and the appended claims, the term “or” is generally employed in its sense including “and/or” unless the content clearly dictates otherwise.

The term “implantable medical device” includes, for example, an implantable signal generator (ISG), a cardiac pacemaker, an implantable defibrillator, a congestive heart failure device, a hearing implant, a cochlear implant, a neurostimulator, a drug pump, a ventricular assist device, an insulin pump, an implantable sensing system, an artificial heart, a bone growth stimulator, or a prosthetic device, and the like.

Examples of “operably coupled” include, but are not limited to, electrically coupled, electrically connected, mechanically coupled, mechanically coupled, electrically and mechanically coupled, electrically and mechanically connected, and capable of being operably coupled.

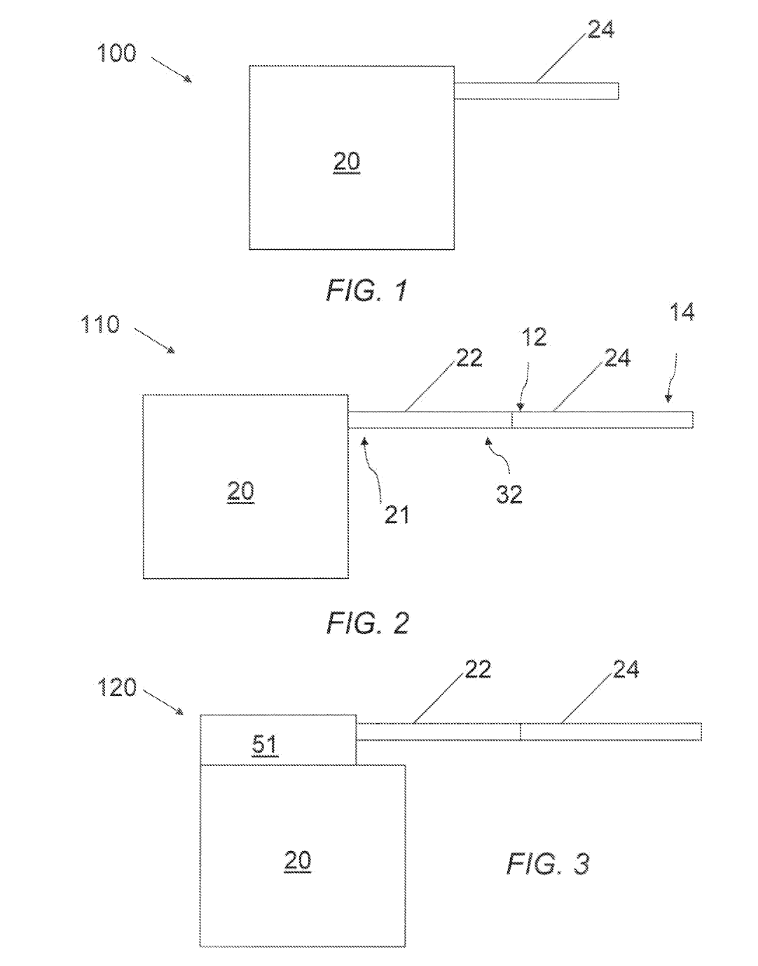

FIG. 1 displays a representation of an implantable medical therapy system 100. The system 100 comprises an ISG 20 and a lead 24 that is operably coupled to the ISG 20. The ISG 20 can be any electrical signal generator or similar implantable medical device useful for delivering therapy to a patient or for patient diagnostics. For example, ISG 20 may be a sensing device hearing implant; a signal generator such as a cardiac pacing device or defibrillator, a neurostimulator (such as a deep brain stimulator, a spinal cord stimulator, a subcutaneous stimulator, etc.), a gastric stimulator; or similar devices. FIG. 2 depicts a system 110 that comprises a lead extension 22 or other adaptor to couple lead 24 to ISG 20. While not shown, it will be understood by one of skill in the art, that more than one lead 24 may be operably coupled to one ISG 20 or one extension 22, or that more than one extension 22 may be operably coupled to one ISG 20. Generally, the lead extension 22 has a proximal end 21 coupled to the ISG 20, and a distal end 32 coupled to the lead 24.

FIG. 3 displays another exemplary therapy system 120 that includes an ISG 20 having a connector block 51 connecting it to lead 24, or as displayed in FIG. 3, extension 22 (or lead adaptor) couples the lead 24 to the ISG 20. While not shown, it will be understood by one of skill in the art, that lead 24 may be coupled to ISG 20 through connector block 51 without extension 22 or an adaptor.

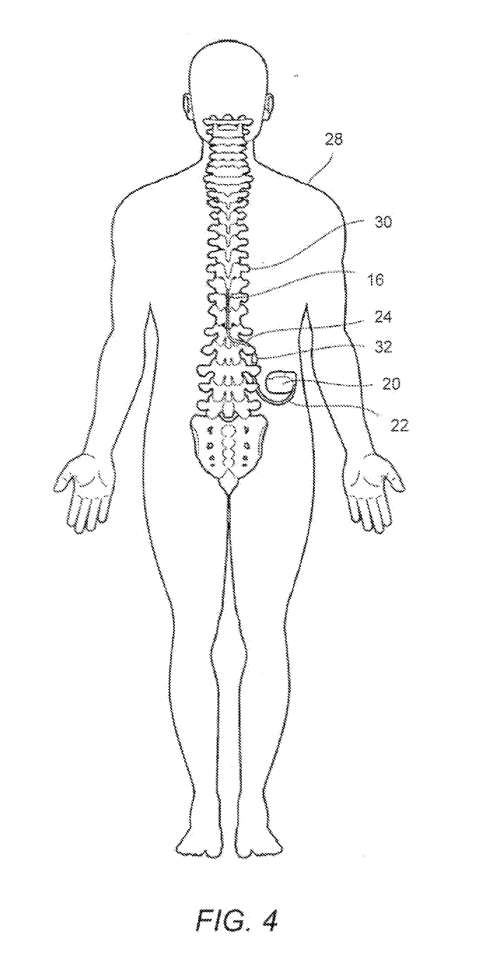

FIG. 4 is a schematic diagram of an implantable medical device 20 implanted within a human body or patient 28. The implantable medical device 20 is illustrated as an implantable signal generator (ISG) however; the implantable medical device 20 may be any implantable medical device as described above and can be placed in any location within a patient or on the surface of a patient's skin.

As seen in FIG. 4, an ISG 20 can be utilized with a lead extension 22 having a proximal end 21 coupled to the ISG 20, and a lead 24 having a proximal end coupled to a distal end 32 of the lead extension 22; and a distal end of the lead 24 that includes at least one electrode 16. In other embodiments, the proximal end of the lead 24 is coupled directly to the ISG 20, without using a lead extension 22. When a lead is referred to herein as connecting to an ISG, it will be understood by one of skill in the art that the lead can be connected to the ISG or the lead extension can be connected to the ISG. It will also be understood that portions of this disclosure that refer to components of the lead connecting to the ISG 20 can be referring to components of the lead or components of the lead extension connecting to the ISG 20.

The ISG 20 can be implanted in any useful region of the body such as in the abdomen of a patient 28. Similarly, the lead 24 can be implanted at any other useful region in the body, such as somewhere along the spinal cord 30. It will also be understood that a lead 24 as referred to herein can be modified to be used with other types of implantable medical devices and still be within the present disclosure.

The leads and ISGs discussed herein can be utilized as part of an implantable therapy delivery system, examples of which were illustrated in FIGS. 2, 3, and 4. Such implantable therapy delivery systems include at least one lead, an optional lead extension, an optional adaptor, and an implantable medical device, such as an implantable signal generator (ISG). Although portions of this specification refer to an ISG, one of skill in the art will also understand that other implantable medical devices can also be utilized herein.

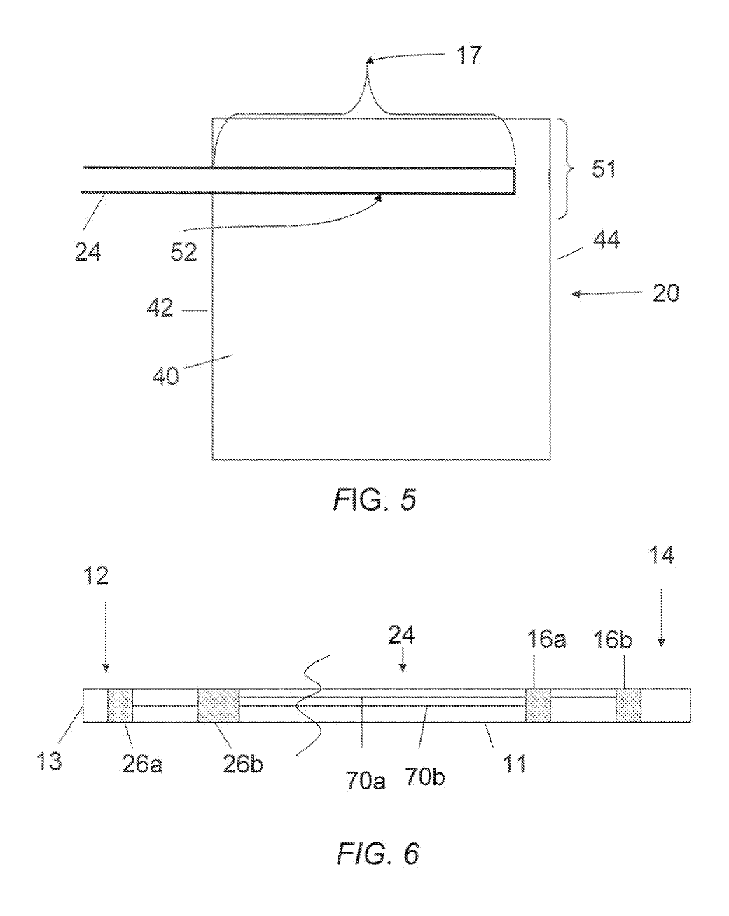

One embodiment of an ISG 20 is depicted in FIG. 5. The ISG 20 includes a housing 40, and an operably coupled connector block 51. The connector block 51 can be separate from the housing 40 but may be permanently or releasably operably coupled to the housing 40, or the connector block 51 can be integral with the housing 40 and can be a designated portion of the housing 40 (depicted in FIG. 5). The connector block 51 includes a lumen 52 that extends through the connector block 51 from one surface of the connector block 51. A single ISG 20 can include more than one connector block 51, a connector block can have more than one lumen 52, or both. In one embodiment, the lumen 52 extends through at least a portion of the connector block 51 from the first surface 42. The lumen 52 could also extend through at least a portion of the connector block 51 from the second surface 44. The lumen 52 is generally configured to receive at least a portion of a lead within at least a portion of the lumen 52. The lumen 52 can also be configured to receive more than one lead.

FIG. 5 also illustrates a lead 24 that is operably coupled in the lumen 52 of a connector block 51 of an ISG 20. The figure illustrates a lead 24 having a connection portion 17 of a lead body 11 (discussed further below). As seen there, the connection portion 17 is the portion of the lead 24 that is within the lumen 52 of the connector block 51 when the lead 24 is operably coupled to the ISG 20 through the connector block 51.

FIG. 6 depicts a lead 24 that includes a lead body 11 having a proximal portion 12 with a proximal end 13, and a distal portion 14. The lead body 11 will be discussed further below.

The lead 24 includes at least one electrode 16. In one embodiment, the lead 24 includes a plurality of electrodes 16, illustrated as first electrode 16a, and second electrode 16b in FIG. 6. All multiple components referred to herein are considered to be “first”, “second”, and so on starting with the component that is closest to the proximal end 13 of the lead body. One of skill in the art will understand that the components could be designated starting from the other direction, or any other way, as well. The plurality of electrodes 16 are generally located at the distal portion 14 of the lead body 11. In one embodiment, a lead 24 having at least four electrodes is utilized. In another embodiment, a lead having at least eight electrodes is utilized. One of skill in the art will understand, having read this specification, that any type and combination of electrodes normally utilized can be utilized herein. Examples of such electrodes include, but are not limited to, ring electrodes, coil electrodes, and segmented electrodes.

The lead 24 also includes at least one electrical contact 26. In an embodiment, the lead includes a plurality of electrical contacts 26. The at least one electrical contact 26 is generally located at the proximal portion 12 of the lead body 11. The example depicted in FIG. 6 depicts two electrical contacts, 26a and 26b, but one of skill in the art will understand that any number of electrical contacts can be utilized. One of skill in the art will also understand that the number of electrical contacts is at least partially dictated by the number of electrodes 16 on the distal end of the lead 24. In one embodiment, the lead 24 includes at least four electrical contacts. In another embodiment, a lead having at least eight electrical contacts can be utilized. In one embodiment, a lead has an equal amount of electrodes and electrical contacts. The electrical contacts 26 generally function to electrically connect the lead 24, and more specifically, the at least one electrode 16 of the lead 24 with at least one electrical connector in a connector block of an ISG when the lead is operably connected with the ISG. Materials and methods of manufacturing electrical contacts including those generally utilized by one of ordinary skill in the art can be utilized in manufacturing leads in accordance with this disclosure.

The lead 24 also includes at least one conductive element 70. In an embodiment, the lead includes a plurality of conductive elements. The conductive elements generally function to electrically connect the at least one electrode 16 to the at least one electrical contact 26. The conductive elements are generally located within the lead body 11 and generally traverse the lead body from the distal portion to the proximal portion (which is analogous to traversing the lead body from the proximal portion to the distal portion). The exemplary lead 24 depicted in FIG. 6 shows two conductive elements 70a, and 70b, however, one of skill in the art will understand that any number of conductive elements can be utilized. One of skill in the art will also understand that the number of conductive elements is at least partially dictated by the number of electrodes and electrical contacts. In one embodiment, there are an equal amount of electrodes, electrical contacts and conductive elements. Materials and Methods of manufacturing conductive elements including those generally utilized by one of ordinary skill in the art can be utilized in manufacturing leads in accordance with this disclosure.

In one embodiment, the lead 24 includes a wire having insulation thereon and includes one or more conductive elements 70 each coupled at the proximal end of the lead body 11 to an electrical contact 26 and to electrodes 16 at the distal end of the lead body 11. Leads in accordance with this description can be designed to be inserted into a patient percutaneously, surgically implanted, or other implantation methods. In some embodiments, the lead 24 may contain a paddle at its distal end for housing electrodes 16. In many embodiments, the lead 24 may include one or more ring electrodes at the distal end of the lead 24.

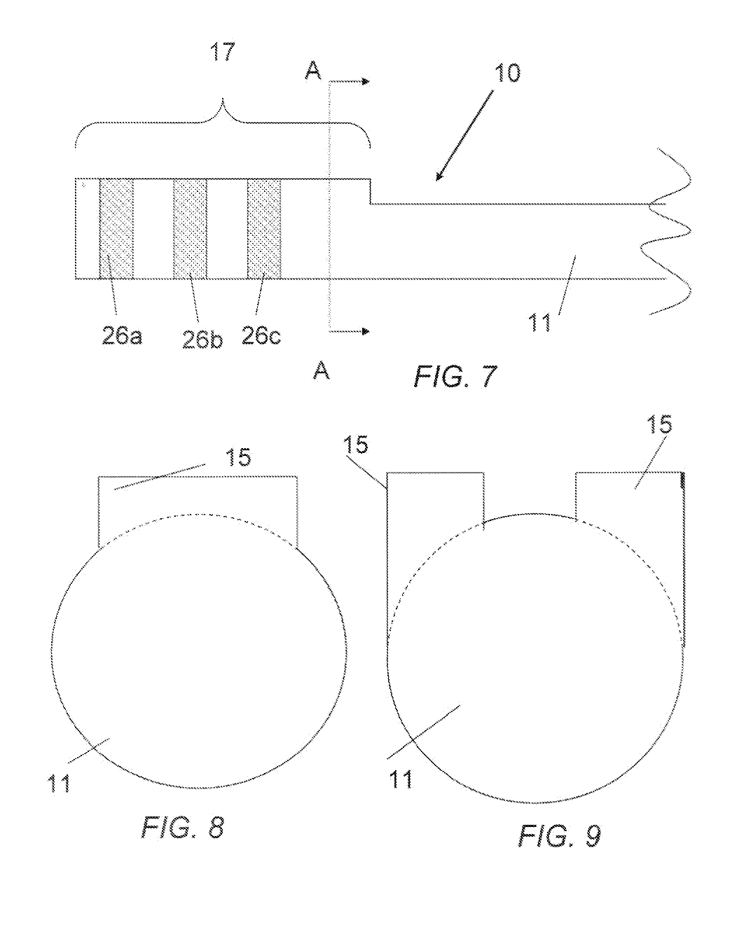

As illustrated in FIG. 7 (and FIG. 5) the lead body 11 includes a connection portion 17. The connection portion 17 of the lead body 11 is the portion that is within the lumen of a connector assembly of an implantable medical device when the lead is operably coupled to the implantable medical device.

A lead that is utilized herein has a lead body with at least the connector portion having an outer surface that is substantially non-circular. Alternatively, an outer surface of the lead body that is substantially non-circular could also be described as the lead body having an axial cross section that is substantially non-circular. In an embodiment, only the connector portion of the lead body has an axial cross section that is substantially non-circular. In an embodiment, the connector portion of the lead body and a portion of the lead body that extends beyond the connector portion toward the distal portion of the lead has an axial cross section that is substantially non-circular. In an embodiment, the entire lead has a lead body that has an axial cross section that is substantially non-circular. In an embodiment, the entire lead as well as a lead extension (if it is utilized) has an axial cross section that is substantially non-circular. In an embodiment, the entire lead, the lead extension, and the lead adaptor has an axial cross section that is substantially non-circular.

An axial cross section that is substantially non-circular, as used herein, includes geometric forms other than circles, including, but not limited to triangles, squares, rectangles, parallelograms, trapezoids, ovals, pentagons, hexagons, and octagons for example. An axial cross section that is substantially non-circular also refers to circles and geometric forms other than circles that include at least one portion that is incongruent with the circle or geometric form. Examples of an incongruous portion include, but are not limited to a recess, a portion that is inside the perimeter of the generally defined geometric form (a circle or other geometric form); a ridge, a portion that is outside the perimeter of the generally defined geometric form (a circle or other geometric form).

FIG. 7 illustrates one example of a longitudinal cross section of a lead body 11 that has an axial cross section at the connector portion 17 that is substantially non-circular. FIG. 8 shows an axial cross section of the representation of FIG. 7 taken along the line A-A in FIG. 7. As seen in FIG. 8, the lead body 11 has an axial cross section at the connector portion 17 that is of a generally circular form and includes at least one portion that is incongruent with the circle. In the embodiment depicted in FIG. 8, the one portion that is incongruent with the circle extends beyond the perimeter of the circle designated by the dashed line. More specifically, the portion that is incongruent with the circle that forms the generally defined form of the lead body at the connector portion can be referred to as a ridge. FIG. 8 refers to the portion as ridge 15. FIG. 9 illustrates another possible cross section of the lead depicted in FIG. 7. As seen there, the axial cross section of the connector portion 17 can be of a generally circular form and include more than one portion that is incongruent with the circle designated by the dashed line. In the example depicted in FIG. 9, it can be seen that there are two ridges 15.

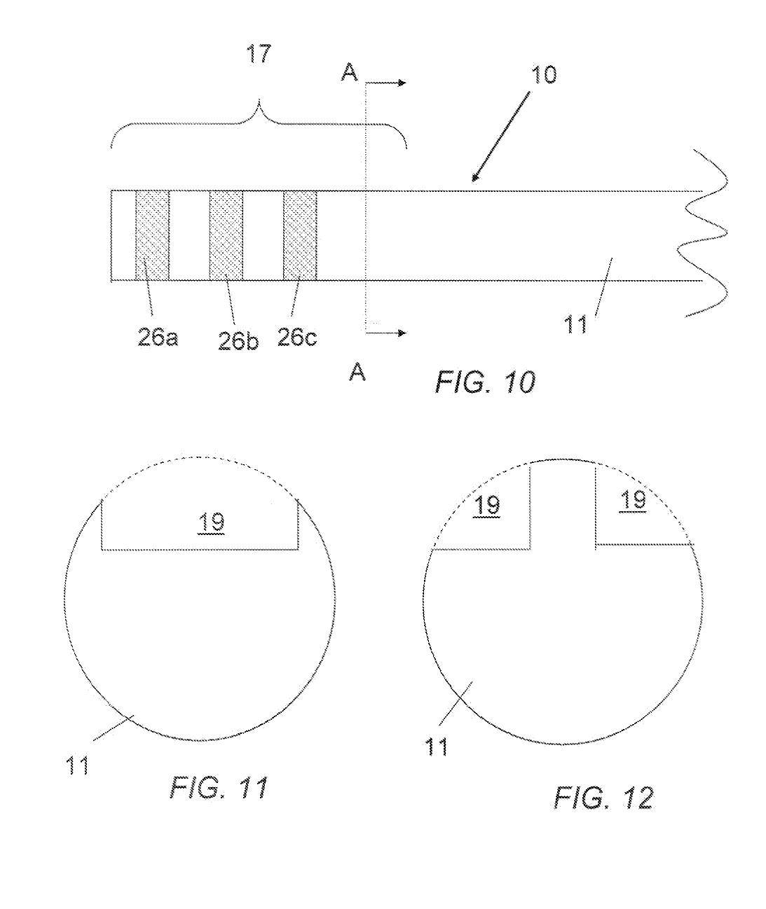

FIG. 10 illustrates another example of a longitudinal cross section of a lead body 11 that has an axial cross section at the connector portion 17 that is substantially non-circular. FIG. 11 shows a cross section of the representation of FIG. 10 taken along the line A-A in FIG. 10. As seen in FIG. 1, the lead body 11 has an axial cross section at the connector portion 17 that is of a generally circular form and includes at least one portion that is incongruent with the circle designated by the dashed line. In the embodiment depicted in FIG. 1, the one portion that is incongruent with the circle extends inside the perimeter of the circle. More specifically, the portion that is incongruent with the circle that forms the generally defined shape of the lead body at the connector portion can be referred to as a recess. FIG. 11 refers to the portion as a recess 19. FIG. 12 illustrates another possible cross section of the lead depicted in FIG. 10. As seen there, the axial cross section of the connector portion 17 can be of a generally circular form and include more than one portion that is incongruent with the circle designated by the dashed line. In the example depicted in FIG. 12, it can be seen that there are two recesses 19.

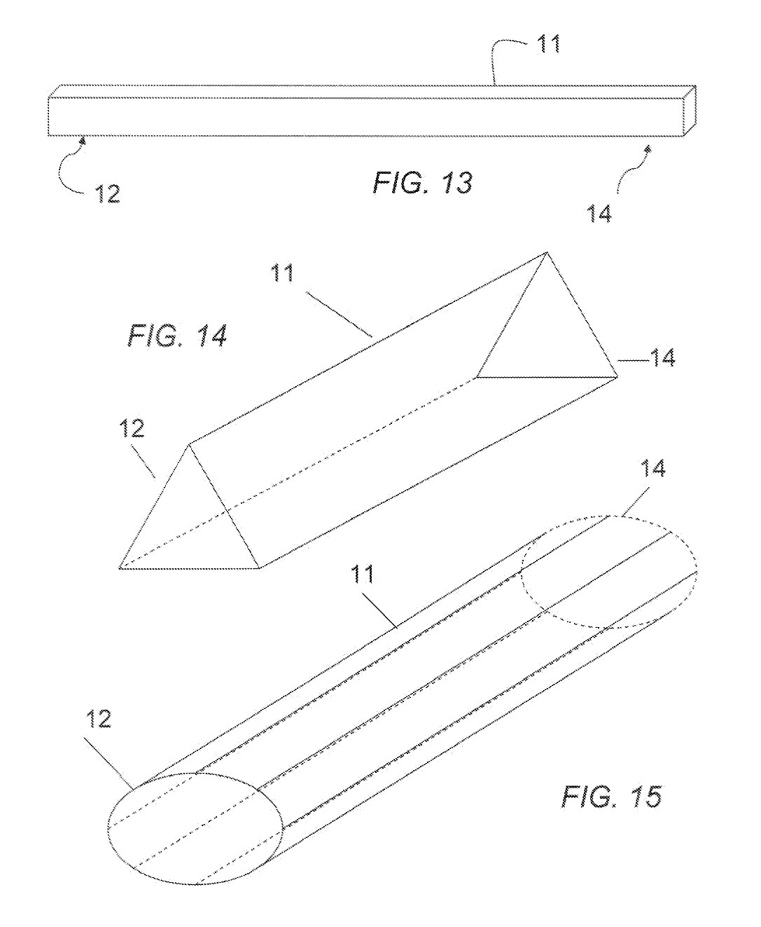

In an embodiment, at least the connector portion of the lead body has a non-circular shape that is a geometric form other than a circle, including, but not limited to triangles, squares, rectangles, parallelograms, trapezoids, ovals, pentagons, hexagons, and octagons. In an embodiment, the axial cross section of the lead body has a shape or the lead body has an axial cross section that is square, oval, or triangle. An embodiment, depicted in FIG. 13 shows a lead that has a lead body 11 that has a square axial cross section along the entire length of the lead. An embodiment depicted in FIG. 14 shows a lead that has a lead body 11 that has a triangle axial cross section along the entire length of the lead. An embodiment depicted in FIG. 15 shows a lead that has a lead body 11 that has an oval axial cross section along the entire length of the lead.

Leads having an axial cross section of at least a connector portion that has a geometric form other than a circle and have a remaining portion of the lead body that has a different axial cross section geometry are also included in this description. An embodiment includes a lead having a connector portion with an axial cross section that has a geometric form other than a circle and the remaining portion of the lead that has an axial cross section that is circular.

Although not depicted, at least the connector portion of the lead body can also generally have a shape other than a circle with at least one portion that is incongruent with its generally defined shape. The axial cross section of the lead body of at least the connector portion can also generally have a shape other than a circle with more than one portion that is incongruent with its generally defined form (also not shown).

One of skill in the art, having read this specification, would be aware of appropriate materials and would know how to manufacture such a lead. Specifically, one of skill in the art would know the materials to be used for the lead body, as well as the other portions of the lead; and would know how to manufacture such leads. Materials that could be utilized to manufacture leads as discussed herein could be similar to those that are utilized in manufacturing commercially available leads, including but not limited to those available from Medtronic, Inc. (Minneapolis, Minn.). Examples of such commercially available leads include, but are not limited to, Model 3487A Pisces—Quad® lead, and Model 3998 Specify® lead. Methods of manufacturing the leads would be known to one of skill in the art, and could include, but are not limited to injection molding methods, and extrusion molding methods.

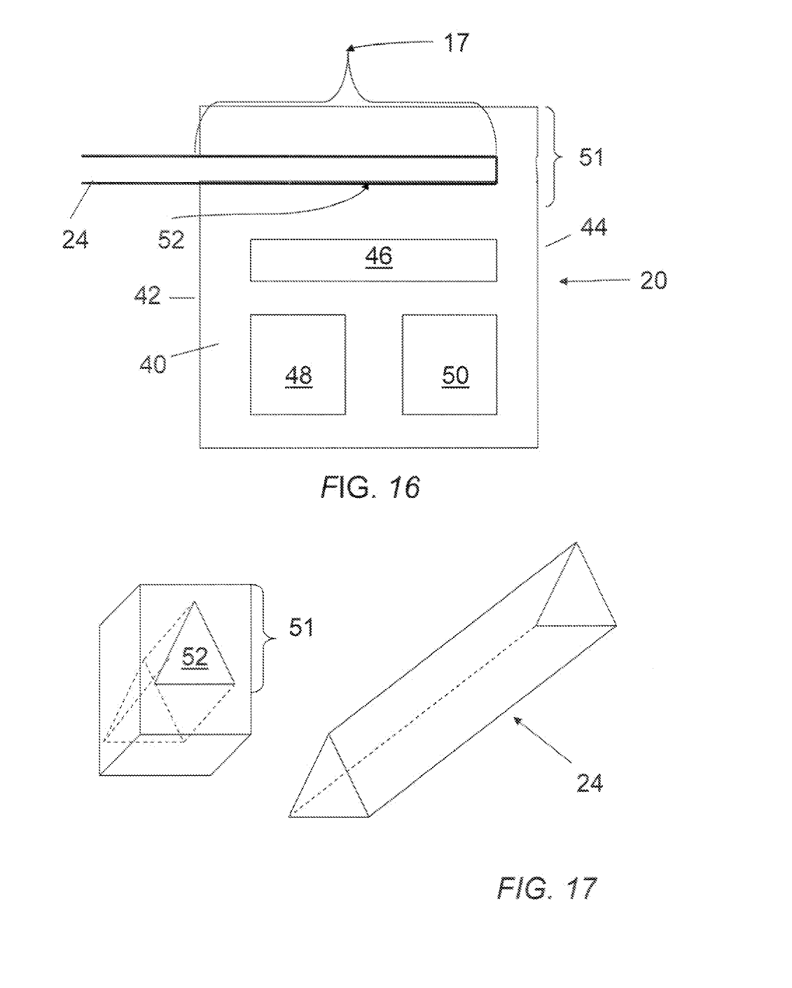

One embodiment of an ISG 20 is depicted in FIG. 16. The ISG 20 depicted in FIG. 16 includes a housing 40. The housing 40 generally functions to contain at least some of the components of the ISG 20. In one embodiment, the housing 40 can generally be of a rectangular type shape. The ISG 20 is operably coupled to the electronic circuitry 46 that generally functions to control the ISG 20. In one embodiment, the electronic circuitry 46 is housed within the housing 40 of the ISG 20 (depicted in FIG. 16). In another embodiment, the electronic circuitry 46 is not contained within the housing 40, but is still operably coupled to the ISG 20. The ISG 20 is also operably coupled to a power source 48. The power source 48 can be contained within the housing 40 (depicted in FIG. 16) or can be outside the housing 40, but still be operably coupled to the ISG 20. The power source 48 can be a battery or an inductive coil, or other such components known to those of skill in the art. In one embodiment, the ISG 20 is also operably coupled to memory 50. In one embodiment, the memory 50 is contained within the housing 40 (depicted in FIG. 16), and in another embodiment, the memory 50 is outside the housing 40 but is still operably coupled to the ISG 20.

The ISG 20 also includes an operably coupled connector block 51. It will also be understood, that a connector block 51, as discussed herein can be a component of a portion of an implantable therapy system other than an implantable medical device or a ISG. For example, a connector block 51, as discussed herein could be part of a lead extension, or an adaptor. The connector block 51 can be separate from the ISG 20 but be permanently or releasably operably coupled to the housing 40, or the connector block 51 can be integral with the housing 40 and can be a designated portion of the housing 40 (depicted in FIG. 16). The connector block 51 includes a lumen 52 that extends through the connector block 51 from one surface of the connector block 51 to another surface of the connector block 51. A single ISG 20 can include more than one connector block 51, a connector block can have more than one lumen 52, or both. In one embodiment, the lumen 52 extends through the connector block 51 from the first surface 42 to the second surface 44. The lumen 52 is generally configured to receive at least a portion of a lead within at least a portion of the lumen 52. The lumen 52 can also be configured to receive more than one lead.

The housing 40 and the connector block 51 can be made of any material commonly known to those of skill in the art, including but not limited to titanium, and other such metals. In one embodiment, the material that makes up the housing and the connector block are a biocompatible material. Exemplary materials include those that are utilized in implantable signal generators available from Medtronic, Inc (Minneapolis, Minn.). It will also be understood by one of skill in the art that possible configurations and dimensions of the housing 40 of the ISG 20 are generally known and can, but need not be utilized. Exemplary configurations and materials include those that are utilized in implantable signal generators available from Medtronic, Inc (Minneapolis, Minn.). One embodiment of an ISG 20 that can be utilized has a housing 40 and a connector block 51 that are integrally formed. In such an embodiment, the connector block is a separate portion of the housing that contains the lumen. The connector block 51 and the housing 40 can be made of the same type of material, can be made from the same piece of material, or can be made of separate materials and can be operably coupled together. In some embodiments, the connector block is a separate piece that is joined to the housing. In some embodiments, the connector block is a portion of the housing and is only distinguished by being the portion of the housing 40 that contains the lumen 52.

The electronic circuitry 46 that is operably coupled to the ISG 20 can generally be similar to those known to one of skill in the art. Examples of such can be found in implantable signal generators available from Medtronic, Inc. (Minneapolis, Minn.). The power source 48 of the ISG 20 can also generally be similar to that known to those of skill in the art. Examples of such can be found in implantable signal generators available from Medtronic, Inc. (Minneapolis, Minn.). The memory 50 can generally include any magnetic, electronic, or optical media, such as random access memory (RAM), read-only memory (ROM), electronically-erasable programmable ROM (EEPROM), flash memory, or the like, or a combination thereof. Examples of such can be found in implantable signal generators available from Medtronic, Inc.

The lumen 52 includes at least one electrical connector, which can generally include material configured to provide electrical contact. In an embodiment, the lumen 52 includes a plurality of electrical connectors. In one embodiment, the at least one electrical connector can also mechanically stabilize the lead and/or the electrical contact that the electrical connector is in contact with. Materials and configurations that can be utilized as electrical connectors are known to those of skill in the art. Examples of such configurations include, but are not limited to set screws made of an electrically conductive material, coil springs that can make electrical contact, friction fit contacts (also referred to as wiping contacts or beam contacts), or similar devices. A specific example of a coil spring that can make an electrical contact is a Bal Seal contact ring available from Bal Seal Engineering Co. Inc (Foothill Ranch, Calif.). Other examples of such devices can be found in implantable signal generators available from Medtronic, Inc. (Minneapolis, Minn.) for example. In one embodiment, a combination of more than one type of electrical connector can be housed in the lumen 52. In one embodiment, both set screws and coil springs that make electrical contact can be utilized in a lumen 52.

The lumen 52 has an inner surface and an axial cross section. The at least one electrical connector, and the lumen 52 are configured so that the electrical connector is positioned adjacent the inner surface of the lumen 52. The electrical connectors are configured adjacent the inner surface of the lumen 52 and are configured to make electrical contact with a lead that is inserted into the lumen 52. The inner surface of the lumen 52 has a geometric configuration that is complementary to the outer surface of at least the connector portion of a lead body that is configured to be inserted into the lumen 52. In one embodiment, at least a portion of the lumen 52 has an inner surface that is complementary to the outer surface of the lead body. In one embodiment, the lumen 52 has an inner surface that is complementary to the outer surface along enough of its length to allow the lead to be operably coupled to the connector block.

As used herein, a geometric configuration that is complementary to the outer surface of a least the connector portion of the lead body means that the inner surface of the lumen 52 has a complementary non-circular shape as at least the connector portion of the lead body.

FIG. 17 provides an example of a complementary inner surface of a lumen 52 of a connector block 51 and lead 24. As seen in FIG. 17, the lead has a triangle shape, along its entire length and the inner surface of the lumen 52 has a triangle shape. If the lead 24 were inserted into the lumen 52 (configured with electrical connectors), the lead would operably fit and could electrically connect the lead 24 to the connector block 51. It will also be understood that less than the entire length of the lead body could have the triangle shape as long as the entire length of at least the connector portion has the substantially non-circular shape.

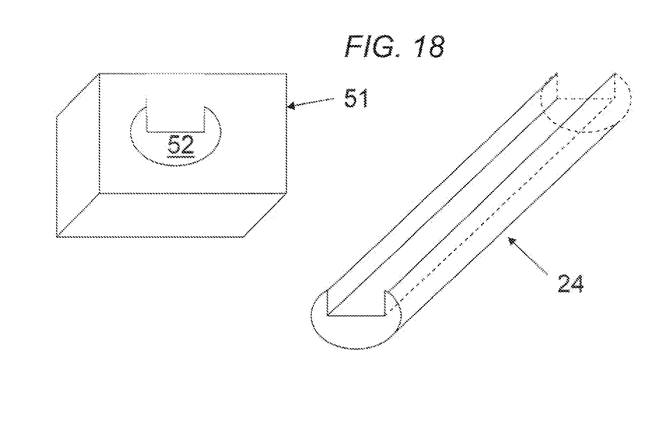

FIG. 18 provides another example of a complementary inner surface of a lumen 52 of a connector block 51 and outer surface of a lead 24. The extension of the lumen 52 in FIG. 18 all the way through the connector block 51 is not shown in order to simplify the figure. One of skill in the art will understand that the lumen 52 will extend through at least a portion of the connector block 51. The lumen 52 shown in FIG. 18 has a portion that extends inside the perimeter of the generally shaped oval lumen. The complementary lead 24 also has a portion that extends inside the perimeter of the generally shaped oval lead body. These portions could also be referred to as recesses. It will also be understood that less than the entire length of the lead body could have the displayed shape as long as the entire length of at least the connector portion has the substantially non-circular shape. It will also be understood that less than the entire length of the lumen could have the displayed shape as long as enough of the lumen has the shape to operably couple the lead.

As with the outer surface of at least the connector portion of the lead body, the inner surface of the lumen 52 has a shape or alternatively the lumen 52 has an axial cross section that is substantially non-circular. Substantially non-circular, as used herein, includes geometric forms other than circles, including, but not limited to triangles, squares, rectangles, parallelograms, trapezoids, ovals, pentagons, hexagons, and octagons. In an embodiment, the inner surface of the lumen has a shape or the lumen has an axial cross section that is square, oval, or triangle. Non-circular also refers to circles and geometric forms other than circles that include at least one portion that is incongruent with the circle or geometric form. Examples of an incongruous portion include, but are not limited to a recess, a portion that is inside the perimeter of the generally defined geometric form (a circle or other geometric form); a ridge, a portion that is outside the perimeter of the generally defined geometric form (a circle or other geometric form).

In an embodiment, an implantable medical system includes a lead having at least a connector portion with an axial cross section that is substantially non-circular; and a connector block having a lumen with an inner surface that has a configuration that is complementary to the outer surface of the connector portion of the lead body. A lead having at least a connector portion with an axial cross section that is substantially non-circular is also disclosed. A connector block having a lumen with an axial cross section that has a substantially non-circular shape is also disclosed.

The complementary nature of the outer surface of at least the connector portion of the lead and the inner surface of the lumen of the connector block forms a mechanical lockout that can enhance the likelihood that only MRI safe components are utilized with other MRI safe components. For example, a connector block that has a lumen with an inner surface that is complementary to a lead having an outer surface of at least the connector portion that includes a recess or a geometric form other than a circle would not be able to be operably coupled to a lead with out that recess or geometric form. Such a configuration could ensure that leads other than the desired leads, i.e. MRI safe leads, would not be able to be implanted and utilized in an incorrect system.

The non-circular axial cross section of the lead body and the non-circular axial cross section of the lumen of the connector block could also offer a visual indicator that a component is of a particular variety, for example, an MRI safe lead and connector block. In an embodiment, a lead that has a non-circular axial cross section on a larger portion than the connector portion may provide advantages for visual indication of a particular lead characteristic (i.e. MRI safety). In an embodiment, a lead that has a non-circular axial cross section along its entire length may provide advantages for visual indication of a particular lead characteristic (i.e. MRI safety) and may also be able to ensure that only other MRI safe components, such as lead extensions or adaptors can be operably coupled thereto. In an embodiment, a lead that has a non-circular axial cross section along its entire length is utilized with a lead extension having a non-circular axial cross section and optionally a lead adaptor having a non-circular axial cross section.

Such a system can also optionally include a physician programmer and a patient programmer (not shown). In one embodiment, the ISG 20 can include an implantable signal generator of the type available from Medtronic, Inc., which is generally capable of generating multiple pulses occurring either simultaneously or one pulse shifting in time with respect to the other, and having independently varying amplitudes and pulse widths. The ISG 20 is operably coupled to a power source and the electrical circuitry for sending precise, electrical pulses to the patient to provide a desired treatment or therapy. While the ISG 20, in many embodiments, provides electrical stimulation by way of pulses, other forms of stimulation may be used such as continuous electrical stimulation.

Also disclosed is a method of connecting a MRI safe lead to an implantable medical device wherein the lead and connector block are as discussed above, and the method includes inserting the proximal end of the lead into the lumen of the connector block; and determining that the lead is MRI safe, wherein the lead is MRI safe if the entire connector portion of the lead will operably fit within the lumen, and the lead is not MRI safe if the entire connector portion of the lead will not operably fit within the lumen. If the lead is MRI safe, it can then be operably connected to the implantable medical device. If the lead is not MRI safe, a different lead can be inserted into the lumen.

Thus, embodiments of an implantable medical electrical lead and connector block are disclosed. One skilled in the art will appreciate that the present disclosure can be practiced with embodiments other than those disclosed. The disclosed embodiments are presented for purposes of illustration and not limitation.

What is claimed is:

1. An implantable medical system comprising:
a. an implantable electrical lead comprising:
i. a lead body having an outer surface, a proximal portion, a distal portion, and a connector portion wherein at least the connector portion of the lead body has an axial cross section that is substantially non-circular;
ii. at least one electrode located at the distal portion of the lead body;
iii. at least one electrical contact located within the connector portion of the lead body; and
iv. at least one conductive element that electrically couples the at least one electrode to the at least one electrical contact; and
b. a connector block that is configured to be operably coupled to the implantable electrical lead, wherein the connector block comprises:
i. a lumen comprising an inner surface and having an axial cross section; and
ii. at least one electrode connector positioned adjacent the inner surface of the lumen,
wherein the at least one electrode connector is configured to be electrically coupled to the at least one electrical contact of the lead, and
wherein the inner surface of the lumen has a configuration that is complementary to the outer surface of at least the connector portion of the lead body.

2. The implantable medical system according to claim 1, wherein more than the connector portion of the lead body has an axial cross section that is non-circular.

3. The implantable medical system according to claim 1, wherein the entire lead body has an axial cross section that is non-circular.

4. The implantable medical system according to claim 1, wherein the axial cross section of the connector portion of the lead body generally defines a geometric form and comprises at least one portion that is incongruent with the generally defined geometric form.

5. The implantable medical system according to claim 4, wherein the at least one portion that is incongruent with the generally defined geometric form is inside the perimeter of the generally defined geometric form.

6. The implantable medical system according to claim 5, wherein the axial cross section of the lumen generally defines the same geometric form as the axial cross section of at least the connector portion of the lead body and comprises at least one portion that is incongruent with the generally defined geometric form.

7. The implantable medical system according to claim 6, wherein the at least one portion of the axial cross section of the lumen that is incongruent with the generally defined geometric form extends beyond the perimeter of the generally defined geometric form.

8. The implantable medical system according to claim 5, wherein the generally defined geometric form is a circle.

9. The implantable medical system according to claim 1, wherein the axial cross section of the connector portion of the lead body generally defines a triangle, a square, a rectangle, a parallelogram, a trapezoid, an oval, a pentagon, a hexagon, an octagon, or a star.

10. The implantable medical system according to claim 9, wherein the axial cross section of the lumen generally defines a triangle, a square, a rectangle, a parallelogram, a trapezoid, an oval, a pentagon, a hexagon, an octagon, or a star.

11. The implantable medical system according to claim 9, wherein the entire lead body has an axial cross section that defines a triangle, a square, a rectangle, a parallelogram, a trapezoid, an oval, a pentagon, a hexagon, an octagon, or a star.

12. The implantable medical system according to claim 1, wherein the axial cross section of the connector portion of the lead body generally defines a triangle, a square, or an oval

13. An implantable electrical lead comprising:
a lead body having an outer surface, a proximal portion, a distal portion, and a connector portion wherein at least the connector portion of the lead body has an axial cross section that is substantially non-circular;
at least one electrode located at the distal portion of the lead body;
at least one electrical contact located at the proximal portion of the lead body; and
at least one conductive element that electrically couples the at least one electrode to the at least one electrical contact.

14. The implantable electrical lead according to claim 13, wherein the entire length of the lead body has an axial cross section that is substantially non-circular.

15. The implantable electrical lead according to claim 13, wherein the axial cross section of the connector portion of the lead body generally defines a geometric form and comprises at least one portion that is incongruent with the generally defined geometric form.

16. The implantable electrical lead according to claim 15, wherein the at least one portion that is incongruent with the generally defined geometric form is inside the perimeter of the generally defined geometric form.

17. The implantable electrical lead according to claim 15, wherein the generally defined geometric form is a circle.

18. The implantable electrical lead according to claim 13, wherein at least the connector portion of the lead body has an axial cross section that is a triangle, a square, a rectangle, a parallelogram, a trapezoid, an oval, a pentagon, a hexagon, an octagon, or a star.

19. The implantable electrical lead according to claim 13, wherein the axial cross section of the connector portion of the lead body generally defines a triangle, a square, or an oval

20. An implantable medical device comprising:
a connector block that is configured to be operably coupled to an implantable electrical lead, wherein the connector block comprises:
i. a lumen comprising an inner surface and having an axial cross section that is substantially non-circular, and being configured to allow an implantable electrical lead to be inserted; and
ii. at least one electrode connector positioned adjacent the inner surface of the lumen, and configured to electrically couple a lead that is inserted into the lumen.

21. The implantable medical device according to claim 20, wherein the implantable medical device is a lead extension, an implantable signal generator, or a lead adaptor.

22. The implantable medical device according to claim 20, wherein the axial cross section of the lumen generally defines a geometric form and comprises at least one portion that is incongruent with the generally defined geometric form.

23. The implantable medical system according to claim 22, wherein the at least one portion of the axial cross section of the lumen that is incongruent with the generally defined geometric form extends beyond the perimeter of the generally defined geometric form.

24. The implantable medical device according to claim 20, wherein the axial cross section of the lumen generally defines a triangle, a square, a rectangle, a parallelogram, a trapezoid, an oval, a pentagon, a hexagon, an octagon, or a star.
